(19) United States
(12) Patent Application Publication
(10) Pub. No.: US 2005/0059017 A1
(43) Pub. Date: Mar. 17, 2005

(54) System and method for extending dynamic range of a detector

(76) Inventors: Mark F. Oldham, Los Gatos, CA (US); Austin B. Tomaney, San Francisco, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/660,110

(22) Filed: Sep. 11, 2003

(51) Int. Cl. C12Q 1/68; G01J 5/02

(52) U.S. Cl. 435/6; 250/339.02

(57) ABSTRACT

A system and method for measuring signals having a wide range of intensity components using detectors adapted for use in biological analysis devices. In certain biological analysis applications, signals emitted by a sample may have intensity components that vary over several orders of magnitude. Measurement of such a signal may yield an acceptable quality for one intensity component at the expense of another component. For example, a detector configured to measure a relatively weak intensity component may cause it to overflow when subjected to a relatively strong intensity component. The detector can be adapted to be operated at different configurations to allow measurements of different components of the signal, and the results can be combined to yield an accurate representation of the signal.

### BACKGROUND

1. Field

The present teachings generally relate to the field of signal processing and more particularly, to a system and methods for extending the effective dynamic range of detectors associated with biological analysis systems.

2. Description of the Related Art

During biological analysis, such as nucleotide sequencing or microarray processing, photo-detectors may be used to detect signals arising from labeled samples or probe features responsive to selected target analytes. These signals may take the form of electromagnetic emissions that are desirably analyzed to quantify signal intensities arising from each labeled sample or probe feature and are subsequently resolved to quantitatively or qualitatively evaluate the presence of a target analyte within a sample.

In certain biological analysis applications, the target analytes may be present in the sample with a wide range of relative abundances, and it may be desirable to accurately measure the relative abundance of each analyte. For example, a sample of nucleotides may have relative abundances having a range that extends several orders of magnitude. Such a sample can then yield electromagnetic emissions having intensities that also has a range extending several orders of magnitude.

Photo-detectors typically have limitations on the range of electromagnetic signals that could be measured accurately. For example, measurements of relatively low intensity signals can have problems associated with a low signal-to-noise ratio. Also, measurements of relatively high intensity signals can have problems associated with an upper limit of what the detector can handle. Thus, measurement of a signal having a wide range of intensity components can be problematic in biological analysis systems. Consequently, there is an ongoing need for an improved approach to the manner in which photo-detectors are used in biological analysis systems.

### SUMMARY

One aspect of the present teachings relates to a system for interrogating a sample using one or more probes configured to be responsive to sample particles. The one or more probes generates one or more identifiable signals following interaction with the sample particles. The sample composition is resolved, at least in part, by identifying the signals identifiable signals comprise a first signal component indicative of a relative abundance of a first particles and a second signal component indicative of a relative abundance of a second particles. The system comprises a detector configured to detect at least a portion of the one or more identifiable signals associated with the constituent probes of the one or more probes. The position of each constituent probe and the signal arising therefrom are used to identify the presence or absence of particles contained within the sample. The detector is configured to operate at different configurations that result in different detector output signals in response to the one or more identifiable signals. The system further comprises a controller configured to control the detector's operating configuration such that the detector can be operated at a first configuration and a second configuration. The first configuration is adapted to measure the first signal component in an effective manner and the second configuration is adapted to measure the second signal component in an effective manner. The controller is further configured to combine the measurements of the first and second signal components at their respective first and second configurations so as to yield a representation of the one or more identifiable signals that includes the first and second signal components. The detector's ability to be operated at the first and second configurations facilitate an improved identification of the presence or absence of particles contained in the sample when the range of relative abundances of the particles is relatively large.

In certain embodiments, the detector comprises a charge-coupled device (CCD) having an array of pixels. Each pixel is adapted to collect charge in response to the one or more identifiable signals. The pixel has an upper limit on the amount of charge it can collect. In one embodiment, the amount of charge collected for a given intensity of the identifiable signal is generally proportional to the duration of collection. The amount of charge collected for a given duration is generally proportional to the intensity of the intensity of the identifiable signal. In one embodiment, the first configuration comprises a short duration T1 of charge collection and the second configuration comprises a long duration T2 of charge collection such that the short duration T1 allows collection of charge associated with a relatively strong intensity component of the identifiable signal and the long duration T2 allows collection of charge associated with a relatively weak intensity component of the sufficient charge to be collected as a result of the weak component. Such a value of T2 may result in the strong component to exceed the upper limit on the amount of collectable charge. In one embodiment, the value of the strong component at the long duration T2 can be approximated by scaling the value of the strong component measured at the short duration T1 thereby allowing representation of the strong component of the identifiable signal at a value that exceeds the upper limit. In one embodiment, the strong component from the T1 collection is scaled by a value given by a ratio of T2/T1.

In certain embodiments, the detector comprises a charge multiplier adapted to receive the detectable signal at a cathode and in response emit photoelectrons that are multiplied by a gain and supplied to an anode. The gain depends on the charge multiplier's operating voltage V raised to a selected power. The charge multiplier has a usable range of gain values. In one embodiment, the charge multiplier comprises a photomultiplier tube (PMT). The output signal comprises the charge supplied to the anode. In one embodiment, the charge multiplier comprises a charge intensifier. The anode comprises a phosphor screen that emits electromagnetic radiation from a localized area thereon in response to the receipt of the multiplied electrons. In one embodiment, the charge intensifier further comprises a CCD that detects the localized emission of the electromagnetic energy from the phosphor screen.

In certain embodiments of the charge multiplier, the first configuration comprises the multiplier operated at a first voltage V1 so as to result in a first gain. The second configuration comprises the multiplier operated at a second voltage V2 so as to result in a second gain. In one embodiment, the first voltage V1 comprises a low voltage selected to allow effective measurement of a strong component of the detectable signal. The second voltage V2 comprises a high voltage selected to allow effective measurement of a weak component of the detectable signal. In one embodiment, the value of the high voltage V2 is selected to allow sufficient gain of photoelectrons resulting from the weak component. Such a value of V2 may result in the strong component to result in the strong component to exceed an upper limit associated with the usable range of gain values. In one embodiment, the value of the strong component at the high voltage V2 can be approximated by scaling the value of the strong component of the identifiable signal at a value that exceeds the upper limit. In one embodiment, the representation N1′ of the strong component at the high voltage V2 scale is approximated by a relation log(N1′)=m log(V2/V1) where m represents a slope of a curve obtained by plotting the multiplier's gain versus the voltage in a log-log manner.

Another aspect of the present teachings relates to a method for improving the measurement of one or more types of specific particles of a sample using a detector of a biological analysis system. The specific particles are adapted to emit identifiable signals based on the interaction of the specific particles with corresponding probes. The identifiable signals are captured by the detector to yield an output signal. The detector is adapted to be operated at different configurations that respond differently to the identifiable signals. The method comprises performing a first measurement of the identifiable signals with the detector at a first configuration such that the detector yields a first output signal. The first configuration allows effective measurement of a first type of the specific particles. The method further comprises performing a second measurement of the identifiable signals with the detector at a second configuration such that the detector yields a second output signal. The second configuration allows effective measurement of the second type of the specific particles. The method further comprises combining the first and second output signals to obtain a representation of the identifiable signals. The representation of the identifiable signals includes effective representations of the first and second types of the specific particles to thereby allow improved identification of the specific particles within the sample.

In certain implementations, the first measurement at the first configuration is adapted to effectively measure a relatively strong component of the identifiable signals associated with the first type of the specific particles having a relatively high abundance. The second measurement at the second configuration is adapted to effectively measure a relatively weak component of the identifiable signals associated with the second type of the specific particles having a relatively low abundance. Combining the first and second output signals comprises scaling the first output signal to a scale associated with the second configuration such that the based on the second configuration, the weak component is effectively measured and the strong component is effectively represented based on the strong component allows effective representation of both weak and strong components when a dynamic range associated with the detector is limited and would not be able to measure the strong component at the second configuration.

In certain embodiments, the detector is a charge-coupled device and the first configuration comprises a short exposure duration T1 selected to effectively measure the strong component of the identifiable signals. The second configuration comprises a long exposure duration T2 selected to effectively measure a weak component of the identifiable signals. The scaling of the first output signal comprises multiplying the first output signal value by a ratio T2/T1.

In certain embodiments, the detector is a charge multiplier and the first configuration comprises a low operating voltage V1 selected to effectively measure the strong component of the identifiable signals. The second configuration comprises a high operating voltage V2 selected to effectively measure a weak component of the identifiable signals. The scaling of the first output signal comprises determining the scaled value N1′ of the first output signal based on a relationship log(N1′)=m log(V2/V1) where m represents a slope of a curve obtained by plotting the multiplier's gain versus the voltage in a log-log manner. In one embodiment, the charge multiplier comprises a photomultiplier tube. In one embodiment, the charge multiplier comprises a charge intensifier.

Yet another aspect of the present teachings relates to a method extending the effective dynamic range of a detector that measures detectable signals from a sample undergoing a biological analysis. The detectable signals comprise two or more components representative of two or more components of the sample. The method comprises obtaining a first output signal from the detector operated at a first configuration that allows effective measurement of a first component of the detectable signals. The method further comprises obtaining a second output signal from the detector operated at a second configuration that allows effective measurement of a second component of the detectable signals. The second configuration is such that the first component of the detectable signals would fall outside the detector's dynamic range at the second configuration. The method further comprises scaling the first output signal to a scale associated with the second configuration. The amount of representation of the first output signal at the second configuration thereby extending the effective dynamic range of the detector. Such extension of the effective dynamic range allows improved characterization of the sample having a relatively large range of relative abundances of the two or more components.

In certain implementations, the first configuration is adapted to effectively measure a strong component of the detectable signals. The second configuration is adapted to effectively measure a weak component of the detectable signals. Scaling the first output signal allows representation of both weak and strong components when the dynamic range associated with the detector is limited and would not be able to measure the strong component at the second configuration.

In certain embodiments, the detector is a charge-coupled device and the first configuration comprises a short exposure duration T1 selected to effectively measure the strong component of the detectable signals. The second configuration comprises a long exposure duration T2 selected to effectively measure a weak component of the detectable signals. The scaling of the first output signal comprises multiplying the first output signal value by a ratio T2/T1.

In certain embodiments, the detector is a charge multiplier and the first configuration comprises a low operating voltage V1 selected to effectively measure the strong component of the detectable signals. The second configuration comprises a high operating voltage V2 selected to effectively measure a weak component of the detectable signals. The scaling of the first output signal comprises determining the scaled value N1′ of the first output signal based on a relationship log(N1′)=m log(V2/V1) where m represents a slope of a curve obtained by plotting the multiplier's gain versus the voltage in a log-log manner. In one embodiment, the charge multiplier comprises a photomultiplier tube. In one embodiment, the charge multiplier comprises a charge intensifier.

### BRIEF DESCRIPTION OF THE DRAWINGS

FIG. 1A illustrates a functional block diagram of a system adapted to measure components associated with biological related processes;

FIGS. 1B and C illustrate exemplary biological analysis systems that utilize photodetectors to detect signals from samples adapted to emit electromagnetic energy in a selected manner;

FIGS. 2A and B illustrate a response of an exemplary detector wherein a range of desirable output in response to an input can define the detector's dynamic range;

FIG. 3 illustrates an exemplary detector adapted to measure an exemplary intensity distribution of signals from an exemplary biological sample platform, showing that the range of intensities can vary by several orders of magnitude;

FIGS. 4A-C illustrate functional block diagrams of a detector adapted to operate at two different configurations to allow effective measurements of weak and strong intensity components of signals emitted from the biological sample;

FIGS. 5A-D illustrate an exemplary charge collection process in an exemplary charge-coupled device (CCD) pixel, showing that a collection potential well has a limit on its capacity to hold charge therein, wherein such a limit can define the CCD's dynamic range;

FIG. 6A illustrates an exemplary upper limit of the CCD's charge storage capacity, and how such a limit can be reached by detection of different intensity signals during different exposure times;

FIG. 6B illustrates a process for operating the CCD at two different configurations to allow effective measurements of two different intensity components of signals emitted from the biological sample;

FIG. 6C illustrates one possible configuration of a CCD based detector adapted to facilitate the process of FIG. 6B;

FIG. 7 illustrates an exemplary photomultiplier tube (PMT) adapted to detect signals from a biological sample;

FIG. 8A illustrates an exemplary response of the PMT, showing that a desirable linear response range can define the PMT's dynamic range;

FIG. 8B illustrates a typical gain of the PMT as a function of the PMT's operating voltage;

FIG. 9A illustrates exemplary curves for two PMT output signals as a function of operating voltage, wherein the two curves correspond to strong and weak components of the sample signal;

FIG. 9B illustrates a process for operating the PMT at two different configurations to allow effective measurements of two different intensity components of signals emitted from the biological sample;

FIG. 10 illustrates an exemplary charge intensifier adapted to detect signals from a biological sample;

FIG. 11 illustrates one possible embodiment of a charge intensifier based detector adapted to allow operation of the charge intensifier at different operating configurations;

FIGS. 12A and B illustrate some possible alternate manners in which the PMTs can be used to facilitate measurements of strong and weak signal components; and

FIG. 13 illustrates an exemplary processing of a signal from a biological sample, showing how scaling of one signal from one configuration to a scale associated with the other configuration allows representation of the sample signal that varies in intensity more than what the detector at a given configuration can handle.

### DETAILED DESCRIPTION OF CERTAIN EMBODIMENTS

These and other aspects, advantages, and novel features of the present teachings will become apparent upon reading the following detailed description and upon reference to the accompanying drawings. In the drawings, similar elements have similar reference numerals.

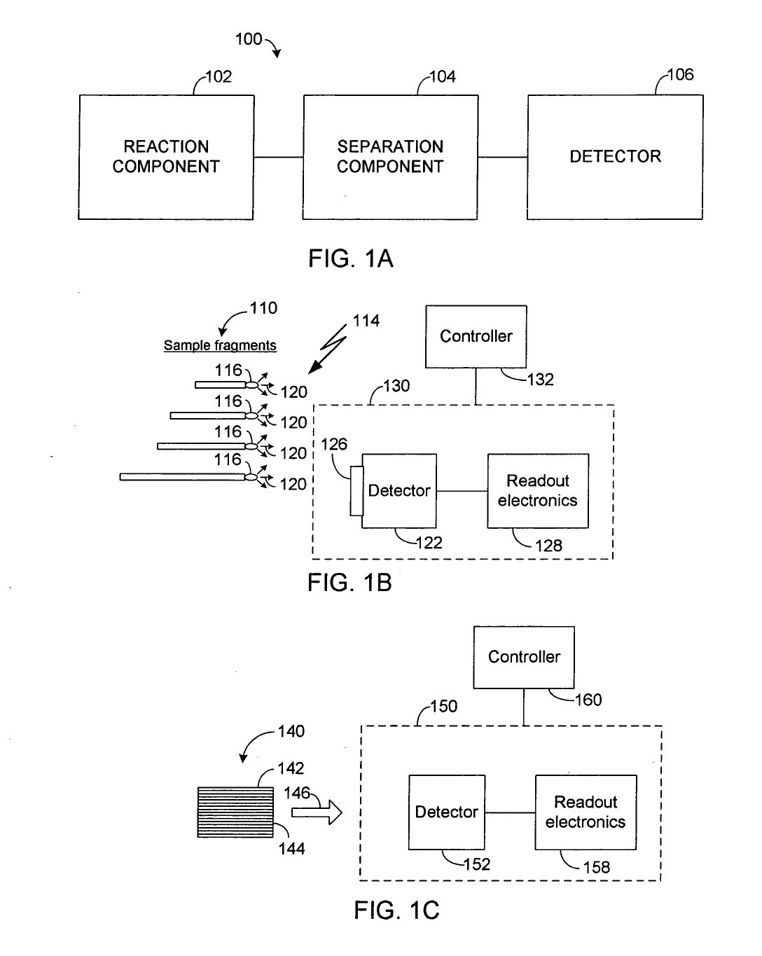

FIG. 1A illustrates an exemplary schematic diagram for a biological analyzer 100 capable of sequence determination or fragment analysis for nucleic acid samples. In various embodiments, the analyzer 100 may comprise one or more components or devices that are used for labeling and identification of the sample and may provide means for performing automated sequence analysis. The various components of the analyzer 100, described in greater detail hereinbelow, may comprise separate components or a singular integrated system. It will be appreciated that the present teachings may be applied to both wherein some of the sequence analysis operations are manually performed. Additionally, the methods described herein may be applied to other biological analysis platforms to improve the overall quality of the analysis.

In various embodiments, the methods and systems of the present teachings may be applied to numerous different types and classes of photo and signal detection methodologies and are not necessarily limited to CCD-based detectors. Additionally, although the present teachings are described in various embodiments in the context of sequence analysis, these methods may be readily adapted to other devices/instrumentation and used for purposes other than biological analysis.

It will also be appreciated that the methods and systems of the present teachings may be applied to photo-detectors in general for a variety of applications, some of which are listed as examples above. Photo-detectors in general convert incident photons to electrical signals, and may include, by way example, CCDs, photomultipliers, or semiconductor based devices such as photo-diodes.

In the context of sequence analysis, the exemplary sequence analyzer 100 may comprise a reaction component 102 wherein amplification or reaction sequencing (for example, through label or marker incorporation by polymerase chain reaction) of various constituent molecules contained in the sample is performed. Using these amplification techniques, a label or tag, such as a fluorescent or radioactive dideoxy-nucleotide may be introduced into the sample constituents resulting in the production of a collection of nucleotide fragments of varying sequence lengths. Additionally, one or more labels or tags may be used during the amplification step to generate distinguishable fragment populations for each base/nucleotide to be subsequently identified. Following amplification, the labeled fragments may then be subjected to a separation operation using a separation component 104. In one aspect, the separation component 104 comprises a gel-based or capillary electrophoresis apparatus which resolves the fragments into substantially discrete populations. Using this approach, electrical current may be passed through the labeled sample fragments which have been loaded into a separation matrix (e.g. polyacrylamide or agarose gel). The application of an electrical current results in the migration of the sample through the matrix. As the sample migration progresses, the labeled fragments are separated and passed through a detector 106 wherein resolution of the labeled fragments is performed.

In one aspect, the detector 106 may identify various sizes or differential compositions for the fragments based on the presence of the incorporated label or tag. In one exemplary embodiment, fragment detection may be performed by generation of a detectable signal produced by a fluorescent label that is excited by a laser tuned to the label's absorption wavelength. Energy absorbed by the label results in a fluorescence emission that corresponds to a signal measured for each fragment. By keeping track of the order of fluorescent signal appearance along with the type of label incorporated into the fragment, the sequence of the sample can be discerned. A more detailed explanation of the sequencing process is provided in commonly assigned U.S. Pat. No. 6,040,586, entitled “Method and System for Velocity-Normalized Position-Based Scanning” which is hereby incorporated by reference in its entirety.

FIG. 1B illustrates exemplary components for a detector assembly 130 which may be used to acquire the signal associated with a plurality of labeled fragments 110. As previously indicated, the labeled fragments 110 may be resolved by measuring the quantity of fluorescence or emitted energy generated when the fragments 110 are subjected to an excitation source 114 of the appropriate wavelength and energy (e.g. a tuned laser). The energy emissions 120 produced by a label 116 associated with the fragments 110 may be detected using a detector 122 as the fragments 110 pass through a detection window 126 wherein one or more energy detecting elements capture at least a portion of the emitted energy from the label 116. In one aspect, an electronic signal is generated by the detector 122 that is approximately proportional to the relative abundance of the fragments 110 passing through the detection window 126 at the time of energy capture and the order which the fragments 110 appear in the detection window 126 may be indicative of their relative length with respect to one another.

A readout electronics assembly 128 is configured to perform readout operations to acquire the electronic signal generated by the detector 122 in response to the fragments 110. In various embodiments, some of the information that may be determined through signal readout and subsequent resolution and peak identification may include determination of the relative abundance or quantity of each fragment population. Evaluation using various known base sequence resolution techniques. It will further be appreciated by one of skill in the art that the exemplified signal distribution may represent one or more nucleic acid fragments for which the relative abundance of each fragment may be evaluated based, in part, upon the determination of the relative area of an associated peak in the signal distribution. The present teachings may therefore be integrated into existing analysis approaches to facilitate peak evaluation and subsequent integration operations typically associated with sequence analysis.

In various embodiments, the readout of the signal from the detector 122 and selected control of the detector 122 may be advantageously performed by a controller 132. The controller 132 may be configured to operate in conjunction with one or more processors and/or one or more other controllers. Such controller and processor's components may include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables. Furthermore, the controller 132 may output a processed signal or analysis results to other devices or instrumentation where further processing may take place.

FIG. 1C illustrates another configuration of exemplary components for a detector assembly 150 which may be used to acquire the signals associated with a plurality of labeled fragments forming an array, microarray, or biochip assay. One exemplary configuration of an array used in biological analysis may comprise a plurality of labeled fragments configured to adhere selectively to an array of tips 144 of a plurality of fibers 142. Such an array type of sample platform 140 may be utilized to simultaneously characterize concentrations of different types of fragments present in a sample. As previously indicated, the labeled fragments attached to the fiber tips 144 may be resolved by measuring the quantity of fluorescence or emitted energy generated when the fragments are subjected to an excitation source of the appropriate wavelength and energy (e.g. a tuned laser). The energy emissions 146 produced by a label associated with the fragments may be detected using a detector 152 that capture at least a portion of the emitted energy from the labeled fragments. In one aspect, an relative abundance of the fragments in the sample being measured.

A readout electronics assembly 158 is configured to perform readout operations to acquire the electronic signal generated by the detector 152 in response to the fragments. In various embodiments, some of the information that may be determined through signal readout and subsequent resolution and peak identification may include determination of the relative abundance or quantity of each fragment population. The spatial resolution of the detected signal allows determination of the position on the sample platform from which the signal was emitted. Thus, by identifying the type of a fiber associated with that position, one can determine the type of fragments attached thereto. Such information facilitates determination of the sequence or composition of the sample using various known base sequence resolution techniques. It will further be appreciated by one of skill in the art that the exemplified signal distribution may represent one or more nucleic acid fragments for which the relative abundance of each fragment may be evaluated based, in part, upon the determination of the relative area of an associated peak in the signal distribution. The present teachings may therefore be integrated into existing analysis approaches to facilitate peak evaluation and subsequent integration operations typically associated with sequence analysis.

In various embodiments, the readout of the signal from the detector 152 and selected control of the detector 152 may be advantageously performed by a controller 160. The controller 160 may be configured to operate in conjunction with one or more processors and/or one or more other controllers. Such controller and processor's components may include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables. Furthermore, the controller 160 may output a processed signal or analysis results to other devices or instrumentation where further processing may take place.

In one aspect, the present teachings relates to various embodiments of detectors being controlled and operated at different configurations to facilitate analysis of sample signals having a wide range of intensities. One of the limitations that biological known, and as described below, the dynamic range of a detector relates to a range of useful detector output that can somehow be correlated to the input.

In detectors such as a charge-coupled device (CCD), a photomultiplier tube (PMT), and a charge intensifier, the lower limit of the dynamic range is typically determined by the resolution of the detector and/or the natural fluctuation associated with the detector's charge generation process. The upper limit of the dynamic range is typically determined by some limit on the amount of charge a given detector can handle. For example, a CCD's upper limit may be defined by its charge collecting capacity. Also, the CCD's upper limit may also be determined by a anti-blooming threshold level. As is known in the art, accumulation of charge beyond such a threshold level causes additional charge to be drained off to prevent the overflowing pixel from affecting nearby pixels. In another example, a PMT may experience an unreliable overall charge output, or even a breakdown if it is operated at too high of a gain setting.

A readout system for reading out of a detector may also have its own dynamic range. For example, an analog-to-digital converter (ADC) typically has a range of charge values it could handle. Thus, a biological analysis system having a detector and a readout assembly may have an overall dynamic range determined by the dynamic ranges of the detector and the readout assembly. It will be appreciated that in the description herein, “dynamic range” may refer to the dynamic range of the detector or the dynamic range of the detector/readout combination without departing from the spirit of the present teachings.

In various embodiments, some of the information that may be determined through signal (from feature) resolution and peak identification may include determination of the relative abundance or quantity of each fragment population. Thus, detectors configured to facilitate analysis of signals at a wider range allows determination of a wider range of relative abundance or quantity of the fragment population in a given sample. Evaluation of the signals may further be used to determine the sequence or composition of the sample using various known base sequence resolution techniques. It will further be appreciated by one of skill in the art that the exemplified signal distribution may represent one or more nucleic acid fragments for which the relative abundance of each fragment may be evaluated based, in part, The present teachings may therefore be integrated into existing analysis approaches to facilitate peak evaluation and subsequent integration operations typically associated with sequence analysis.

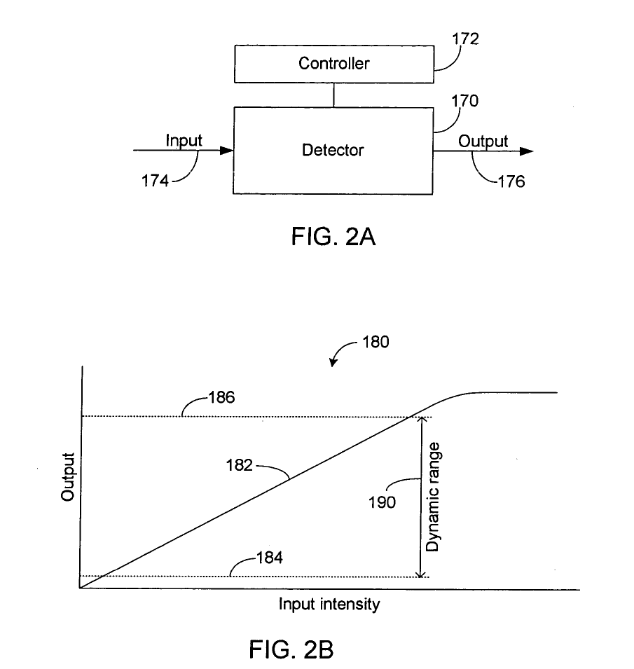

FIG. 2A illustrates a functional block diagram of a detector 170 that is at least partially under the control of a controller 172. In certain embodiments, the controller may be configured to adjust the manner in which the detector 170 operates such that the detector 170 responds differently to an input signal 174. Such different responses of the detector 170 results in an output signal 176 to be at least somewhat different for different detector configurations. Various detectors used in biological analysis applications have different manners in which configurations are adjusted, and several exemplary detector types are described below in greater detail.

FIG. 2B illustrates an exemplary response 180 of a detector at a given operating configuration. The response 180 comprises a curve 182 that shows the detector's output as a function of input signal intensity. In certain embodiments, the curve 182 includes a generally linear response region, as exemplified by a lower 184 and an upper 186 output limits. The lower limit 184 may be determined by the detector's resolution and/or noise associated with the output. The upper limit 186 may be determined by various factors, including but not limited to detector saturation and onset of non-linear response. In FIG. 2B, the curve 182 is depicted as beginning to deviate from its linear relationship above the upper limit 186. In certain embodiments, the range between such exemplary lower and upper limits 184, 186 may be considered to be a dynamic range associated with the detector.

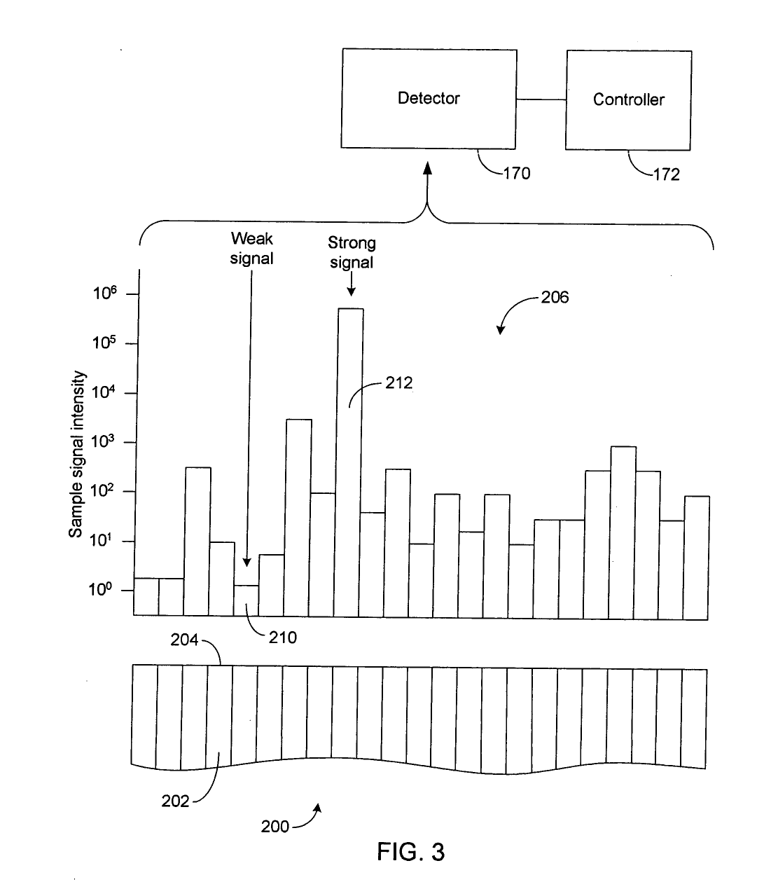

FIG. 3 illustrates an exemplary biological analysis application, showing why it would be desirable to have a detector 170 that has a capability to measure a relatively large range of signal values. An exemplary array type biological sample platform 200 is shown to have a plurality of fibers 202 having ends 204 where various fragments (not shown) adhere to in a selected manner. Such fragments may be tagged with labels that fluoresce when energetically excited. The intensity of electromagnetic signal emitted from a given tip 204 is thus representative of the relative abundance of the tagged fragment at that tip 204.

As shown in FIG. 3, an exemplary intensity distribution 206 corresponding to the array 200 is depicted. The distribution 206 includes an exemplary weak signal 210 and an exemplary strong signal 212. As shown by the logarithmic intensity scale, in certain biological analysis applications there can be a range of relative abundances of fragments (and thus relative intensities) that extends over several orders of magnitudes.

Such a wide range of intensity signals can pose a challenge for a measurement system. For example, if one wishes to perform an accurate measurement of a relatively weak signal, one can increase the sensitivity of a detector. In doing so, a relatively strong signal may exceed the upper limit of the detector's dynamic range. Conversely, if one wishes to perform an accurate measurement of the relatively strong signal, one can decrease the sensitivity of the detector. In doing so, the relatively weak signal may have its measurement value decreased to a level comparable to a noise level, thereby making the weak signal measurement generally unusable.

As previously described, one aspect of the present teachings relates to the detector 170 adapted to operate at different configurations to allow measurements of both relatively weak and strong incident signals. In one aspect, a controller 172 controls the manner in which such different detector configurations are implemented and/or the manner in which measurements from the different configurations are processed to obtain the desired results.

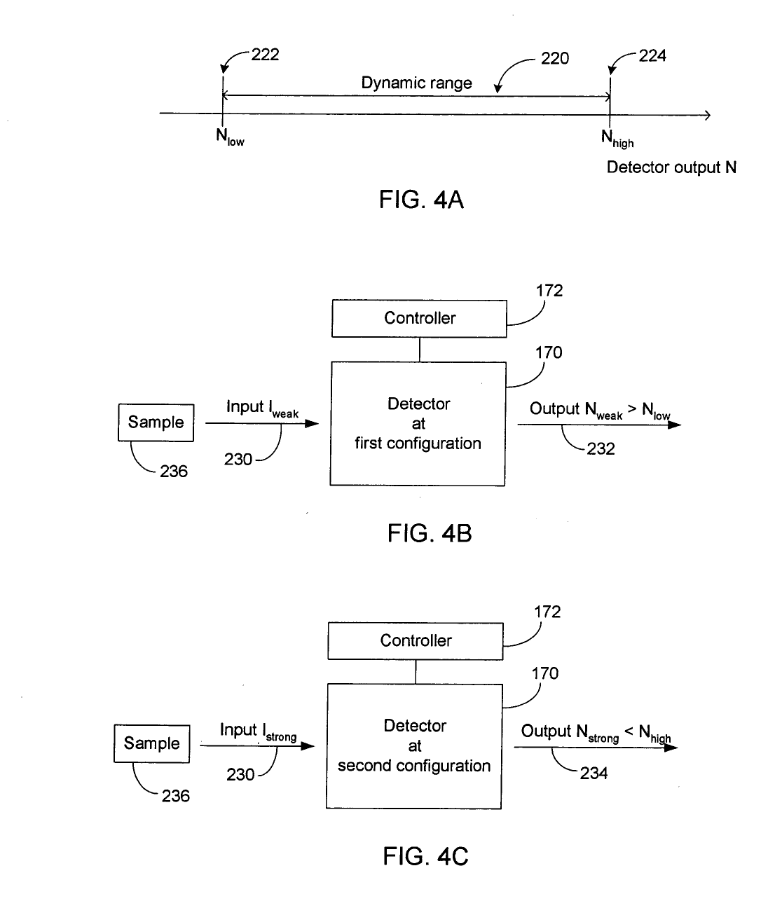

FIGS. 4A-C illustrate functional diagrams of an exemplary dynamic range of a detector and two possible detector configurations that may be implemented to achieve the relatively wide measurable signal range described above. For the purpose of description, the detector 170 is assumed to output a packet of charge N as an output signal. It will be understood that such an output can be converted to various forms of electrical signals for subsequent measurements and/or analysis. Furthermore, it will be appreciated that other detectors applicable to biological analysis devices may output signals in forms other than charge; thus, the concepts disclosed herein may also be used in such detectors without departing from the spirit of the present teachings.

As shown in FIG. 4A, a dynamic range 220 of a detector is a range of values of output N that is useful for analysis. Thus, the exemplary dynamic range 220 is depicted to be between an exemplary lower limit N_{low} 222 and an upper limit N_{high} 224 values of the detector output.

As shown in FIG. 4B, the detector 170 may be operated at a first configuration under the control of the controller 172. Under the first configuration, a signal 230 including a weak component I_{weak} from a sample 236 impinges on the detector 170 and causes the detector 170 to yield an output signal 232 including a weak component N_{weak} that is greater than the lower limit N_{low} 222 of the dynamic range 220.

As shown in FIG. 4C, the detector 170 may be operated at a second configuration under the control of the controller 172. Under the second configuration, the signal 230 including a strong component I_{strong} from the sample 236 impinges on the detector 170 and causes the detector 170 to yield an output signal 234 including a strong component N_{strong} that is less than the upper limit N_{high} 224 of the dynamic range 220.

The operation of the detector 170 in the foregoing manner results in a first and a second set of measurements. The first measurement corresponding to FIG. 4B comprises a useful weak component N_{weak} that is within the dynamic range. Because the detector 170 at the first configuration is sensitive enough for such a measurement of N_{weak}, the strong component of the first measurement may or may not have exceeded the upper limit of the dynamic range. For the purpose of description, it will be assumed that the strong component of the first measurement has exceed the upper limit, and thus information about the strong component has been compromised or lost.

The second measurement corresponding to FIG. 4C comprises a useful strong component N_{strong} that is within the dynamic range. Because the detector 170 at the second configuration has its sensitivity reduced enough for such a measurement of N_{strong}, the weak component of the second measurement may or may not have gone below the lower limit of the dynamic range. For the purpose of description, it will be assumed that the weak component of the second measurement has gone below the lower limit, and thus information about the weak component has been compromised or lost.

One aspect of the present teachings relates to combining the results of the first and second measurements obtained at the first and second detector configurations to yield an approximation of at least one of the weak or strong components that would otherwise be of limited use due to the detector's dynamic range. As an example, one may choose to express the analyzed results of the measurements in terms of the detector sensitivity at its first configuration. Thus, the weak component from the first measurement can represent the value of the weak component. Then, the strong component from the second measurement can be scaled so as to approximate the first configuration sensitivity. Such a scaling of the strong component of the second measurement yield an approximation of the strong component in terms of the first sensitivity, and the valid value of such an approximation exceeds the upper limit of the detector's dynamic range.

In various embodiments, the manner in which the scaling of one measurement can be achieved depends on various operating principles of various detectors. Thus, some of the possible scaling methods are disclosed below by way of examples as various exemplary detectors are described.

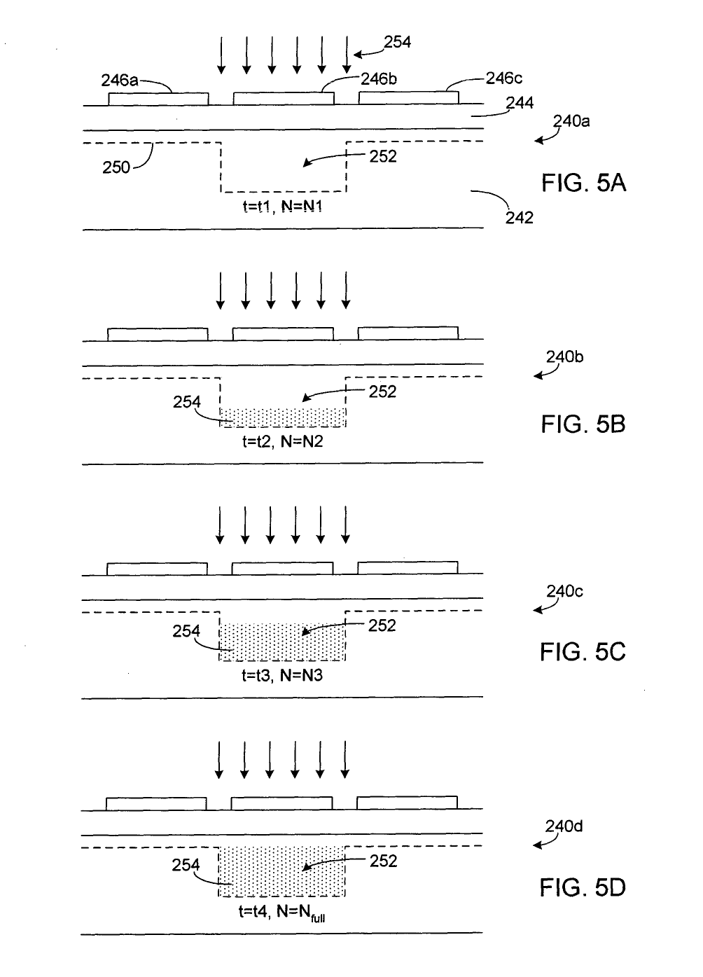

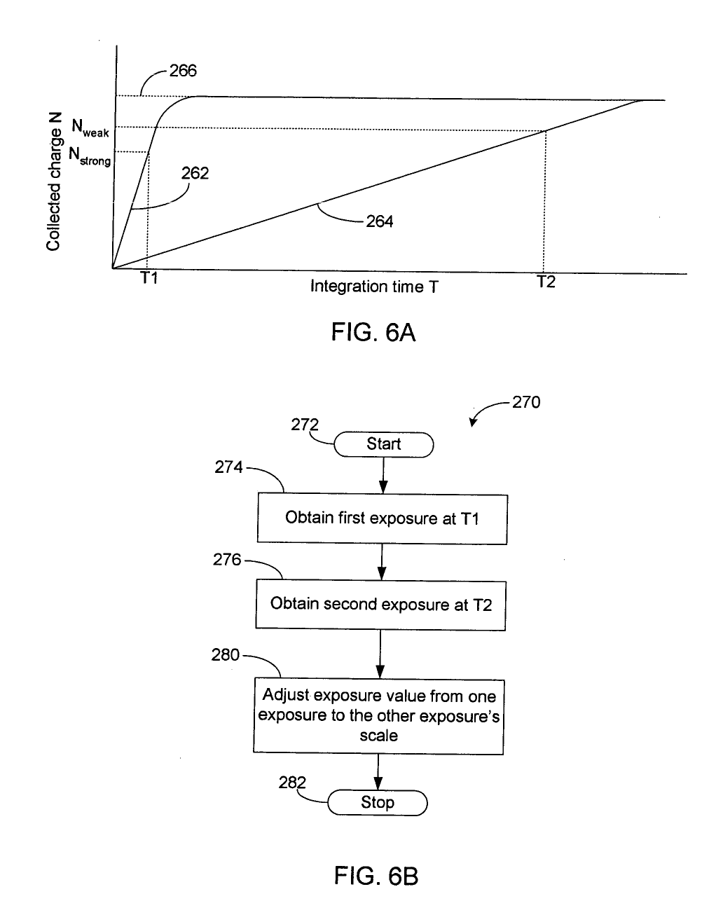

FIGS. 5 to 6 now illustrate a CCD that can be operated at different configurations as described above in a general manner. FIGS. 5A-D illustrate an exemplary charge collection process showing one possible detector parameter that can determine the CCD's dynamic range. The charge collection process at an exemplary pixel is depicted as a series of “snapshots” 240a-d corresponding to FIGS. 5A-D.

As shown in FIG. 5A, the exemplary pixel comprises a plurality of gates 246a-c separated from a substrate layer 242 by an oxide layer 244. Selective doping of the substrate layer in combination of selective application of voltage(s) at the gate(s) 246a-c facilitate formation of a potential profile 250. During the charge collection process, the potential profile 250 comprises a potential well 252 for collecting charge therein. The charge is generated as a result of the pixel's interaction with incident electromagnetic signal 254. As is known in the art, the gate voltages can be altered in a selected sequence to shift the potential well spatially, thereby allowing the collected charge to be transferred out for measurement.

The snapshot 240a of FIG. 5A corresponds to time t=t1, when the collection well 252 is generally empty (N=N1) and ready to receive generated charge. At time t=t2 corresponding to the snapshot 240b of FIG. 5B, charge 254 has accumulated to N=N2. At time t=t3 corresponding to the snapshot 240c of FIG. 5C, charge 254 has accumulated to N=N3. The charge accumulation continues until the collection well becomes “full” (N=N_{full}) of charge 254 at time t=t4, as depicted in the snapshot 240d of FIG. 5D.

In certain embodiments, the “fullness” of the collection well may be determined by an anti-blooming threshold level. As is known in the art, excess charge above this threshold can be drained away to prevent “spillage” into the collection wells of nearby pixels. For the purpose of description, the “fullness” of the pixel's collection well may refer to the anti-blooming threshold level, the potential profile's reference level as depicted in FIG. 5D, and/or any other similar effect(s) that limit the amount of charge collected at the pixel.

The rate of the exemplary charge collection process of FIGS. 5A-D generally depends on the intensity of the incident signal 254. If the incident signal intensity is generally uniform, then the rate of charge collection can be approximated as ΔN/Δt=(N3−N2)/(t3−t2). Such a relationship can also be expressed as
N=cIT (1)
where N represents the number of collected charge, I represents the intensity of the incident signal, c represents a conversion factor between the intensity I and the charge collection rate ΔN/Δt, and T represents an “exposure” time during which charge is collected.

FIG. 6A illustrates two exemplary curves 262 (first) and 264 (second) respectively representative of a strong and weak incident signal intensities. The pixel's charge storing capacity is indicated by a line 266, and for the purpose of description the strong and weak incident signals are assumed to have generally uniform intensities.

The first curve 262 is shown to collect charge at a relatively fast pace so as to reach its collected charge N_{strong} state at an exposure time of T1. The second curve 264 is shown to collect the charge at a relatively slow pace so as to reach its collected charge N_{weak} state at an exposure time of T2.

As shown in FIG. 6A, the integration (exposure) time can be interpreted as being somewhat equivalent to a “gain” of a detector, meaning that it generally determines how much the CCD pixel puts out for a given input condition. If both of the strong and weak signals were to be part of a same signal from a given sample, an exposure of T1 (first exposure) would likely yield a measurement of the weak component that may be buried among the noise level (not shown). Conversely, an exposure of T2 (second exposure) would result in the strong component being truncated at a value associated with the upper limit 266 of the charge storage capacity.

One aspect of the present teachings relates to combining the results of the first and second measurements to yield a combined result having desirable characteristics associated with the weak and strong components of a given signal. For the first and second exposures described above in reference to FIG. 6A, one may choose to select the weak component measurement from the second exposure such that the weak component value is above the noise level by a substantial margin. One may then take the value of the strong component from the first exposure (which is also above the noise level by a substantial margin) and scale it to the “gain” associated with the second exposure. If the intensities of the weak and strong components of the signal remain generally constant during the first and second exposures, the scaling factor can be approximated by a ratio of T2/T1, and the scaled value N′_{strong} can be approximated as N′_{strong}=(T2/T1)N_{strong}.

One way to obtain the first and second exposures for the scaling purpose as described above, is to perform one of the two exposures, followed closely in time by the other exposure. A more detailed explanation of such a method and other possible methods of performing combinations of “short” and “long” exposures are provided in commonly assigned and copending U.S. Patent Application entitled “System and Method For Dynamic Range Extension Using a Variable Length Integration Time Sampling” (application No. 10/271,477) which is hereby incorporated by reference in its entirety.

FIG. 6B illustrates a process 270 that performs the foregoing measurements and scaling. The process 270 begins at a start state 272 and in step 274 that follows, the process 270 obtains a first exposure at a first duration T1. In step 276 that follows, the process 270 obtains a second exposure at a second duration T2. In the description above in reference to FIG. 6A, T1 and T2 were associated with short and long durations respectively. Thus, it will be appreciated that either of the sequences short-long or long-short can be implemented without departing from the spirit of the present teachings. Consequently, steps 274 and 276 can be interchanged in order. Following the two exposures the process 270 in step 280 adjusts the exposure value from one exposure to the other exposure's scale. In the description above in reference to FIG. 6A, the exemplary scaling is done such that the high “gain” value is scaled to the low “gain” scale. It will be appreciated that the reverse (low gain value to high gain scale) scaling can also be performed. Furthermore, the high and low gain values can be scaled to any scale associated with the two exposure T1 and T2 without departing from the spirit of the present teachings. The process 270 ends at a stop state 282.

FIG. 6C illustrates one possible embodiment of a detector 292 configured to facilitate the foregoing method of obtaining two different exposures. The detector 292 may comprise a CCD 294 whose exposure to signals from a sample platform 298 is controlled at least in part by a shutter 296. The shutter 296 can be adapted to be controlled by a controller 290 that is configured to perform the short and long exposures as described above. In such embodiments, an exposure may be defined as a duration for which the shutter remains open.

In certain embodiments, the detector 292 may be configured to operate as a shutterless device, with the exposures being controlled by the application of gate voltages. In such embodiments, an exposure may be defined as a duration for which a charge collection potential remains formed. In either of the shutter or shutterless embodiments or modes, the corresponding exposures can be controlled by the controller 290 so as to facilitate the implementation of concepts disclosed herein without departing from the spirit of the present teachings.

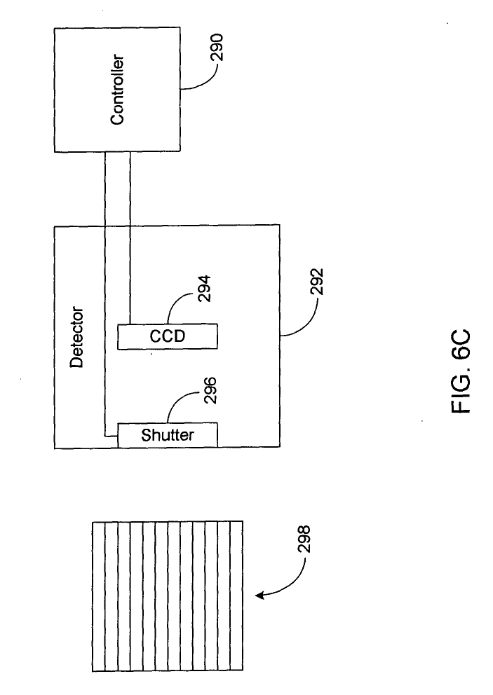

FIGS. 7 to 9 now illustrate a photomultiplier tube (PMT) that can be operated at different configurations as described above in a general manner. FIG. 7 illustrates an exemplary PMT 300 having a cathode 302 and an anode 304 with a plurality of dynodes 306a-c (also referred to as stages) interposed therebetween. Typically, a voltage V_{supply} is applied between the cathode 302 and the anode 304, with the dynodes 306 being held at intermediate potentials via some form of voltage dividing circuit (not shown).

The PMT 300 detects electromagnetic signal 310 from a sample 312 by first converting the electromagnetic signal 310 to photoelectrons 314 at the cathode 302 with some characteristic quantum efficiency. Because the dynode 306a is held as a potential different from that of the cathode 302, the photoelectrons 314 emitted from the cathode 302 accelerate to and strike the first dynode 306a thereby generating additional secondary electrons 316a. The secondary electrons then accelerates to and strike the second dynode 306b thereby generating more secondary electrons 316b. This amplification process continues (the number of dynodes 306a-c exemplary and for the purpose of description) through each of the subsequent stages, and the last dynode (depicted as dynode 306c) ejects a plurality of secondary electrons 316c that are collected by the anode 304 so as to yield a PMT output signal 320.

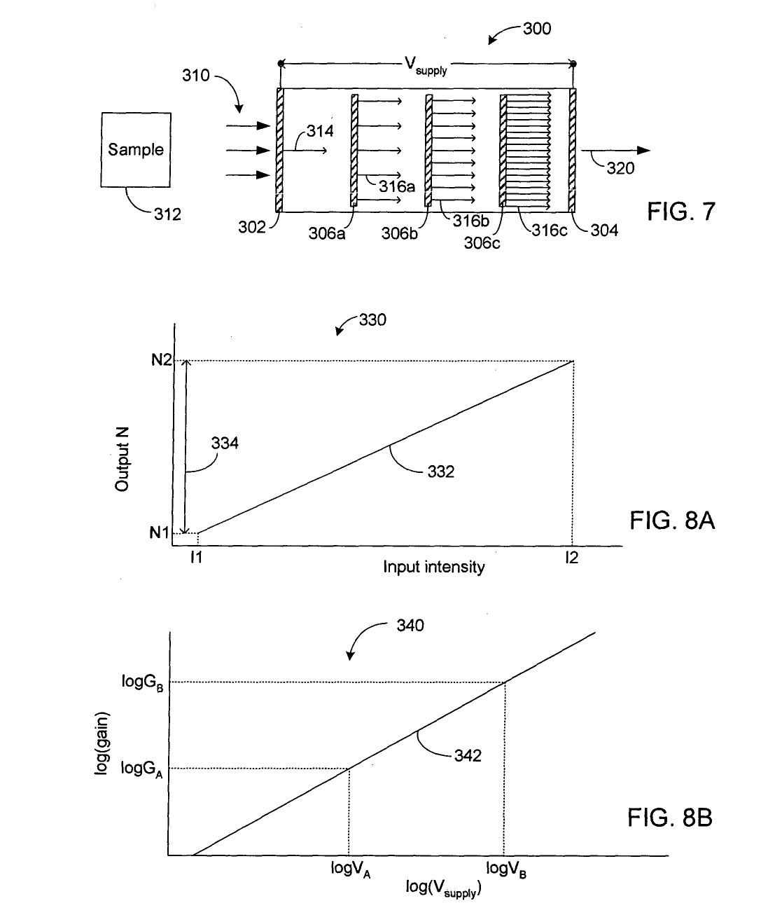

The foregoing PMT charge multiplication can result in a gain of several orders of magnitude, with an actual gain depending on the operating voltage V_{supply}. For a given operating voltage, a PMT typically manifests a generally linear (or at least characterizable in some manner) relationship between its output and the intensity of the input signal. Such a relationship 330 is illustrated in FIG. 8A, where a detector response curve 332 is generally linear in a range between input intensities I1 and I2. Corresponding output values N1 and N2 can then determine one possible dynamic range 334 associated with a PMT.

From FIG. 8A, one can see that an incident signal having an intensity greater than I2 would be outside of the exemplary dynamic range 334. One aspect of the present teachings relates to operating a PMT to facilitate measurement of a signal that would fall outside of a dynamic range of the PMT at one configuration. One possible way of achieving such a result is to use the PMT's operating voltage as a varying parameter, similar to the CCD's exposure time as described above, to obtain measurements of strong and weak signal components at different operating voltages. The two measurements can then be adjusted in a manner described below.

FIG. 8B illustrates an exemplary typical relationship 340 between the PMT's gain and its operating voltage V_{supply} for a given input signal intensity. The gain typically depends on some power of the operating voltage. Thus, a log-log plot of the gain vs. V_{supply} as in FIG. 8B yields a generally linear line 342 with the slope being proportional to the power factor.

As shown in FIG. 8B, for a given PMT, the exemplary curve 342 may be obtained by measuring a given intensity signal at two or more operating voltages (V_{A} and V_{B}) so as to yield two or more gain values (G_{A} and G_{B}). From such measurements, the slope of the line 342 may be determined.

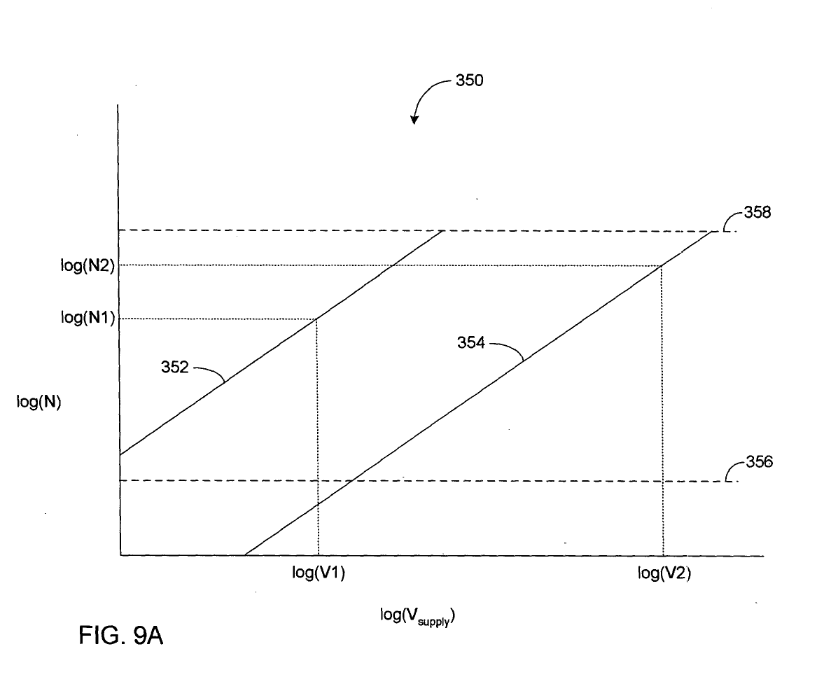

FIGS. 9A-C now describe how the PMT having the foregoing exemplary properties can be operated at two different configurations to allow measurement and combination of the strong and weak signal components. As previously described, it will be assumed for the purpose of description that the strong and weak components' intensities are sufficiently different so that the PMT at one configuration would result in one of the two components being outside of the PMT's dynamic range.

FIG. 9A illustrates an exemplary relationship 350 between the PMT's output N and the operating voltage V_{supply}. A log-log plot yields generally linear curves 352 and 354 corresponding to the strong and weak signal components respectively. As previously described in reference to FIG. 8B, the gain (G) is proportional to voltage to some power. That is, the gain can be expressed as
G=aV^{α} (2)
where a represents a proportionality constant and α represents the power factor. In certain embodiments, the PMT's output N can be approximated as
N=NₚₑG=Nₚₑa V^{α} (3)
where Nₚₑ represents the number of photoelectrons (314 in FIG. 7) ejected from the cathode. Thus, Equation 3 can be expressed as
log(N)=α log(V)+constant. (4)

As shown in FIG. 9A, the strong component of the signal measured at a voltage V1 (first configuration) yields an output N1. The weak component of the signal measured at a higher voltage V2 (second configuration) yields an output N2. For the purpose of description, it will be assumed that the values of N1 and N2 are within the dynamic range defined by limits 356 and 358. It will be understood that although the strong and weak components 352 and 354 are depicted as separate curves in FIG. 9A, the two components may be part of a same signal associated with a sample.

As seen in FIG. 9A, an attempt to measure the strong component (curve 352) at the second voltage V2 would “yield” an output (if somehow measurable) that exceeds the upper limit 358. Also, an attempt to measure the weak component (curve 354) at the first voltage V1 would yield an output below the lower limit 356 and that output may be generally undistinguishable from the noise associated with the operation of the PMT.

One aspect of the present teachings relates to adjusting the output of the PMT obtained at one operating configuration to the scale associated with the other operating configuration. Such a scaling allows analysis of a signal having a relatively large range of intensity components using a PMT having a limited dynamic range.

Thus in the exemplary PMT configurations illustrated in FIG. 9A, one can choose to express the results in terms of the scale associated with the second configuration operated at the higher voltage V2. In such an analysis, one can retain the measured value N2 to represent the weak component, and the strong component can be obtained by scaling the value N1 (obtained at voltage V1). One possible way of scaling the value of N1 to N1′ associated with the higher voltage V2 is as follows. If the strong component line 352 was to be extended to the V2 value, thus yielding N1′ that exceeds the upper limit 356, one can express the slope of the line 352 as
slope=[log(N1′)−log(N1)]/[log(V2)−log(V1)], (5)
from which one can obtain an expression for N1′ as
log(N1′)=(slope)log(V2/V1). (6)
The value of the slope can be determined as described above in reference to FIG. 8B. Thus, the value of N1′ can be determined according to Equation 6.

FIG. 9B illustrates a process 360 that performs the foregoing PMT operations to advantageously obtain an estimation of a signal component at a given configuration. The process 360 begins at a start state 362, and in step 364 that follows, the process 360 obtains a first output at a first operating voltage V1. In step 366 that follows, the process 360 obtains a second output at a second operating voltage V2. In step 370 that follows, the process 360 adjusts the output value from one voltage configuration to the scale associated with the other voltage configuration. In the exemplary scaling method described above in reference to FIG. 9A, the value of the strong signal component was obtained at a relatively low voltage V1 and projected to a value associated with the relatively high voltage V2 scale. The scaling can also be performed the other way—scaling the weak component to the V1 scale without departing from the spirit of the present teachings. The process 360 ends at a stop state 372.

FIG. 9C illustrates a functional block diagram of one possible configuration for operating a PMT 386 that allows the foregoing process (360 of FIG. 9B) to be performed. The PMT 386 is supplied with its operating voltage from a high-voltage (HV) supply 384. In certain embodiments, the output level of the HV supply 384 is controlled by a digital-to-analog converter (DAC) 382 which in turn is controlled by a controller 380. The operation of the PMT 386 in the foregoing manner allows the PMT 386 to detect an electromagnetic signal 390 emitted by a biological sample 392.

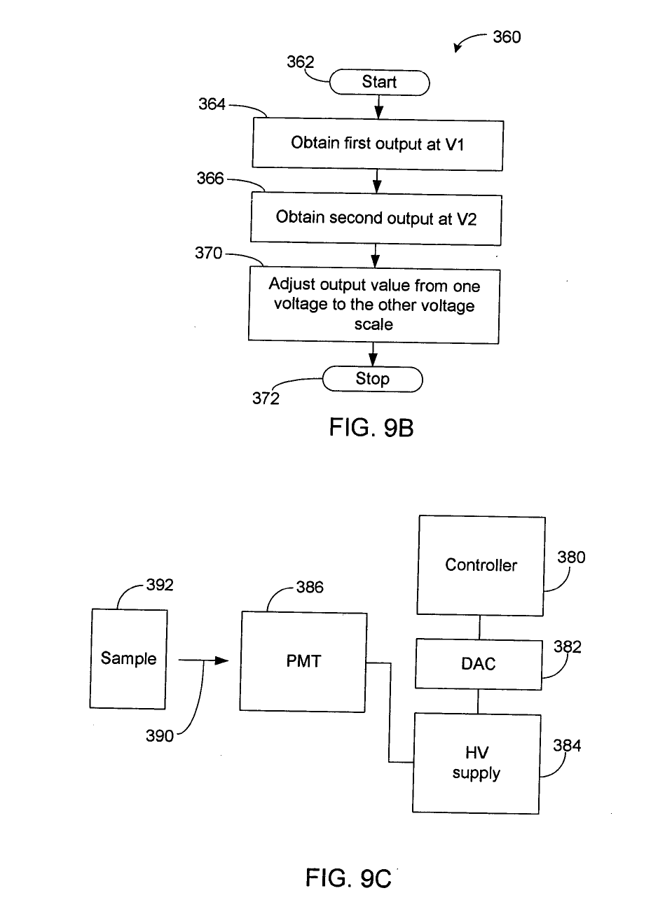

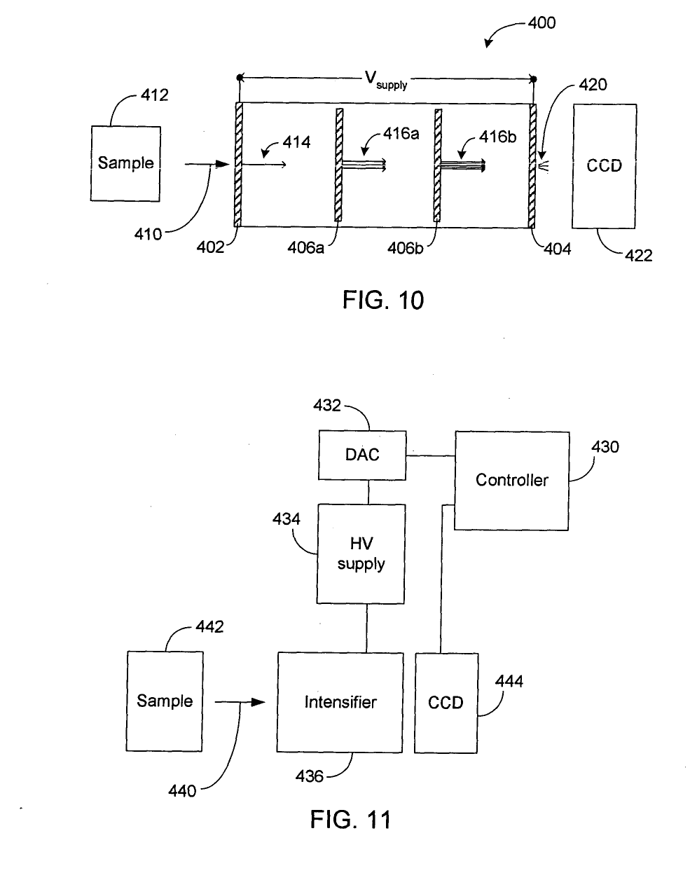

FIGS. 10 to 11 now illustrate a charge intensifier that can be operated at different configurations as described above in a general manner. FIG. 10 illustrates an exemplary intensifier 400 having a cathode 402 and a phosphor screen 404 with a plurality of dynodes 406 interposed therebetween. In certain embodiments, the intensifier operates in a similar manner as that of a PMT, in that an incident electromagnetic signal 410 from a sample 412 ejects primary electron(s) 414 from the cathode 402, and the electrons 414 are multiplied via the dynodes 406 to produce their respective secondary electrons 416. The multiplied electrons 416 then impinge on the phosphor screen 404 at a localized area that can be mapped to the incident location on the cathode 402. Thus, the signal 410 impinging on the cathode 402 results in a plurality of electrons impinging on the corresponding location on the phosphor screen 404 thereby resulting in a localized emission of electromagnetic radiation 420 that can be detected by, for example, a CCD 422.

In certain embodiments, the intensifier 400 is supplied with an operating voltage V_{supply} that facilitates the charge multiplication in a manner similar to that of the PMT. Thus, the operation of the exemplary intensifier 400 at different voltages and advantageous scaling of measured components can be performed in a similar manner as that described above in reference to FIGS. 7-9.

FIG. 11 illustrates a functional block diagram of one possible configuration for operating an intensifier 436 that facilitates the scaling method described above. The intensifier 436 is supplied with its operating voltage from a high-voltage (HV) supply 434. In certain embodiments, the output level of the HV supply 434 is controlled by a digital-to-analog converter (DAC) 432 which in turn is controlled by a controller 430. The operation of the intensifier 436 in the foregoing manner allows the intensifier 436 to detect an electromagnetic signal 440 emitted by a biological sample 442, and to amplify the resulting charge such that the amplified charge can be converted to an electromagnetic signal to be detected by a detector such as a CCD 444. In certain embodiments, the controller 430 may be configured to control the operation of the intensifier 436 and the CCD 444.

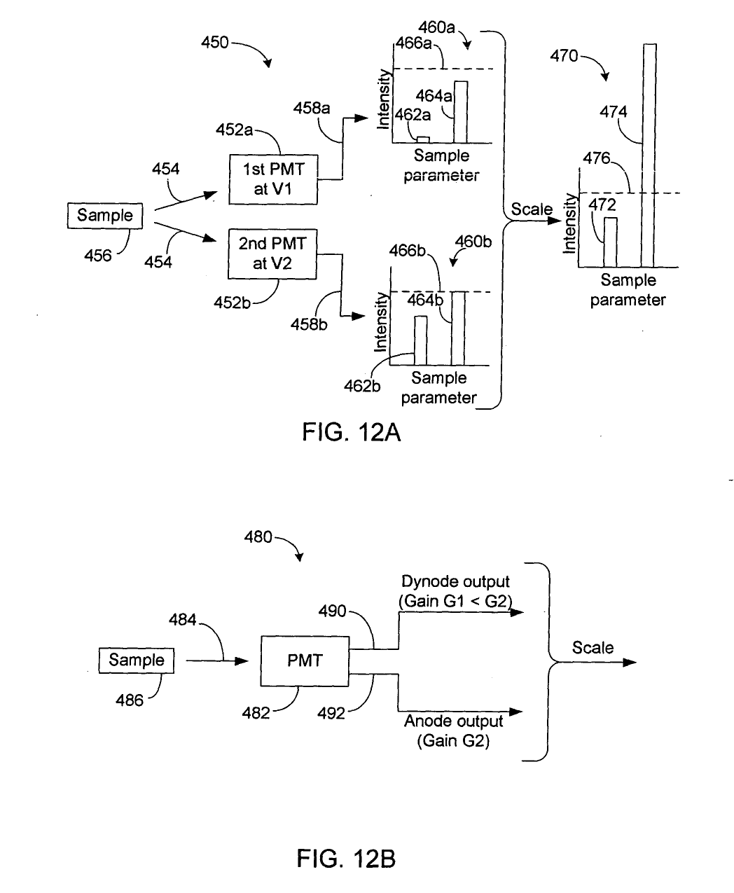

FIGS. 12A-B now illustrate two alternate embodiments of a biological analysis systems that use signals from PMTs as described above to analyze signals having a relatively wide range of intensity components. As described above, the gain of a PMT depends on the operating voltage raised to some power. Consequently, a change in the voltage can have a significant effect on the PMT's gain. Thus, voltage supplies used for PMT operation are typically configured to be relatively stable.

When a voltage setting in such a HV supply is changed, it may take some time before the HV supply and/or the PMT settles to a stable and known voltage configuration. Consequently, the voltage change may limit the pace of operation of the analysis system.

Thus as shown in FIG. 12A, a detector 450 may comprise a first PMT 452a and a second PMT 454b. The first PMT 452a may be configured to operate at a first voltage V1, and the second PMT 452b may be configured to operate at a second voltage V2 that is higher than V1. Thus, signals 454 comprising exemplary strong and weak components from a sample 456 are detected by the first and second PMTs 452a, b. The first PMT 452a yields a first signal 458a that results in an intensity distribution 460a where the strong and weak components are depicted as bars 464a and 462a, respectively. The second PMT 452b yields a second signal 458b that results in an intensity distribution 460b where the strong and weak components are depicted as bars 464b and 462b, respectively. For the two distributions 460a, and b, corresponding upper limits 466a, and b are indicated.

The strong component 464a of the first distribution 460a is shown to be within the dynamic range 466a, and the strong component 464b of the second distribution 460b is shown to be truncated at the upper limit 466b due to the second PMT 452b being operated at its high gain (V2). By scaling the first distribution 460a to the scale associated with the second distribution 460b in a manner described above, one can obtain a scaled distribution 470 where the weak component (bar 472) is represented by a significant value, and the strong component (bar 474) is represented by a relatively large value that exceeds a dynamic range 476.

FIG. 12B illustrates another configuration 480 of a PMT 482 that may be utilized to benefit from the scaling method without having to change the operating voltage. In certain embodiments, the PMT 482 may be configured to allow more than one output therefrom. A first output 490 may comprise a signal from one of the dynodes. A second output 492 may comprise a signal from the anode. As described above, the charge multiplication progresses via each dynode. Thus, an output from a dynode is generally less than that from the anode. Thus, a properly selected dynode can yield the output 490 having a gain of G1 that is less than the anode output gain of G2. The two output signals with different gains G1 and G2 can then be scaled in a manner similar to that described above.

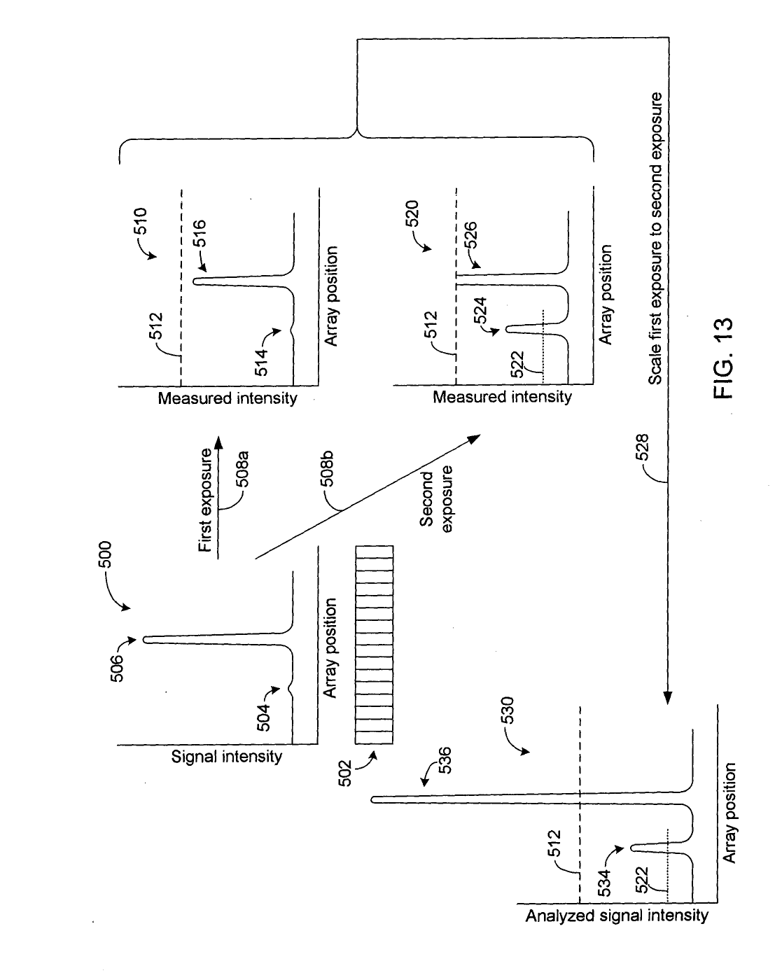

The exemplary operations of the various detectors as described above can be summarized as a generalize signal analysis process illustrated in FIG. 13. An exemplary biological sample platform 502 is depicted to emit signals having an exemplary signal intensity distribution 500. The distribution 500 is shown to comprise an exemplary weak component 504 and a strong component 506, as indicated by their relative peak amplitudes.

Such a signal having the weak and strong components may be detected by a detector operated at a first configuration and at a second configuration. The first configuration yields a first exposure 508a, and the second configuration yields a second exposure 508b. The first configuration is such that the first exposure yields a first measured intensity distribution 510 having a strong peak 516 within an upper limit 512 of a dynamic range. The first measured intensity distribution 510 also includes a weak peak 514 that may or may not be within a lower limit 522 of the dynamic range.

The second configuration is such that the second exposure yields a second measured intensity distribution 520 having a strong peak 526 truncated proximate the upper limit 512, thereby having the strong component information compromised. The second measured intensity distribution 520 also includes a weak peak 524 that extends above the lower limit 522.

The first distribution 510 is then shown to be scaled (as indicated by an arrow 528) to the scale associated with the second distribution 520. A resulting analyzed signal intensity distribution 530 comprises a weak component 534 that is within the dynamic range between the limits 522 and 512. The analyzed distribution 530 further comprises a strong component 536 whose value exceeding the upper limit 512 was approximated by the scaling process. Thus, one can see that foregoing two or more exposures followed by selected scaling allows the sample's relatively wide range of signal component intensities to be captured and analyzed in an improved manner.

Although the above-disclosed embodiments of the present invention have shown, described, and pointed out the fundamental novel features of the invention as applied to the above-disclosed embodiments, it should be understood that various omissions, substitutions, and changes in the form of the detail of the devices, systems, and/or methods illustrated may be made by those skilled in the art without departing from the scope of the present invention. Consequently, the scope of the invention should not be limited to the foregoing description, but should be defined by the appended claims.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

1. A system for interrogating a sample using at least one probe configured to be responsive to the sample wherein the at least one probe generates identifiable signals following interaction with the sample and wherein the sample composition is resolved, at least in part, by identifying the signals associated with the at least one probe and wherein the signals comprise a first signal component indicative of a relative abundance of a first particle species and a second signal component indicative of a relative abundance of a second particle species, the system comprising:
a detector configured to detect at least a portion of the signals associated with the at least one probe wherein the position of each probe and the signal arising therefrom are used to identify the presence or absence of particles contained within the sample and wherein the detector is configured to operate at different configurations that result in different detector output signals in response to the signals; and
a controller configured to control the detector's operational configuration such that the detector can be operated at a first configuration and a second configuration wherein the first configuration is adapted to measure the first signal component in an effective manner and the second configuration is adapted to measure the second signal component in an effective manner and wherein the controller is further configured to combine the measurements of the first and second signal components measured at their respective first and second configurations so as to yield a representation of the signals that includes the first and second signal components.

2. The system of claim 1, wherein the detector's ability to be operated at the first and second configurations facilitate an improved identification of the presence or absence of particles contained in the sample when the range of relative abundances of the particles is relatively large.

3. The system of claim 1, wherein the detector comprises a charge-coupled device (CCD) having an array of pixels wherein each pixel is adapted to collect charge in response to the signals and wherein the pixel has an upper limit on the amount of charge it can collect.

4. The system of claim 3, wherein the amount of charge collected for a given intensity of the signal is generally proportional to the duration of collection and wherein the amount of charge collected for a given duration is generally proportional to the intensity of the intensity of the signal.

5. The system of claim 4, wherein the first configuration comprises a short duration T1 of charge collection and the second configuration comprises a long duration T2 of charge collection such that the short duration T1 allows collection of charge associated T2 allows collection of charge associated with a relatively weak intensity component of the identifiable signal.

6. The system of claim 5, wherein the long duration T2 is selected so as to allow sufficient charge to be collected as a result of the weak component and wherein such a value of T2 may result in the strong component to exceed the upper limit on the amount of collectable charge.

7. The system of claim 6, wherein the value of the strong component at the long duration T2 can be approximated by scaling the value of the strong component measured at the short duration T1 thereby allowing representation of the strong component of the identifiable signal at a value that exceeds the upper limit.

8. The system of claim 7, wherein the strong component from the T1 collection is scaled by a value given by a ratio of T2/T1.

9. The system of claim 1, wherein the at least one probe comprises a plurality of probes forming a probe array.

10. The system of claim 9, wherein the probes are arranged on the probe array in known positions or orientations.

11. The system of claim 1, wherein the detector comprises a charge multiplier adapted to receive the detectable signal at a cathode and in response emit photoelectrons that are multiplied by a gain and supplied to an anode wherein the gain depends on the charge multiplier's operating voltage V raised to a selected power and wherein the charge multiplier has a usable range of gain values.

12. The system of claim 11, wherein the charge multiplier comprises a photomultiplier tube (PMT) having an output signal associated with the charge supplied to the anode.

13. The system of claim 11, wherein the charge multiplier comprises a charge intensifier and wherein the anode comprises a phosphor screen that emits electromagnetic radiation from a localized area thereon in response to the receipt of the multiplied electrons.

14. The system of claim 13, wherein the charge intensifier further comprises a CCD that detects the localized emission of the electromagnetic energy from the phosphor screen.

15. The system of claim 11, wherein the first configuration comprises the multiplier operated at a first voltage V1 so as to result in a first gain and wherein the second configuration comprises the multiplier operated at a second voltage V2 so as to result in a second gain.

16. The system of claim 15, wherein the first voltage V1 comprises a low voltage selected to allow effective measurement of a strong component of the signal and wherein the second voltage V2 comprises a high voltage selected to allow effective measurement of a weak component of the signal.

17. The system of claim 16, wherein the value of the high voltage V2 is selected to allow sufficient gain of photoelectrons resulting from the weak component and wherein such a value of V2 may result in the strong component to result in the strong component to exceed an upper limit associated with the usable range of gain values.

18. The system of claim 17, wherein the value of the strong component at the high voltage V2 can be approximated by scaling the value of the strong component measured at the low voltage V1 thereby allowing representation N1′ of the strong component of the identifiable signal at a value that exceeds the upper limit.

19. The system of claim 18, wherein the representation N1′ of the strong component at the high voltage V2 scale is approximated by a relation log(N1′)=mlog(V2/V1) where m represents a slope of a curve obtained by plotting the multiplier's gain versus the voltage in a log-log manner.

20. A method for improving the measurement of one or more types of specific particles of a sample using a detector associated with a biological analysis system wherein the specific particles are adapted to emit identifiable signals based on the interaction of the specific particles with corresponding probes and wherein the identifiable signals are captured by the detector to yield an output signal and wherein the detector is adapted to be operated at different configurations that respond differently to the identifiable signals, the method comprising:
performing a first measurement of the identifiable signals with the detector at a first configuration such that the detector yields a first output signal wherein the first configuration allows effective measurement of a first type of the specific particles;
performing a second measurement of the identifiable signals with the detector at a second configuration such that the detector yields a second output signal wherein the second configuration allows effective measurement of the second type of the specific particles; and
combining the first and second output signals to obtain a representation of the identifiable signals wherein the representation of the identifiable signals includes effective representations of the first and second types of the specific particles to thereby allow improved identification of the specific particles within the sample.

21. The method of claim 20, wherein the first measurement at the first configuration is adapted to effectively measure a relatively strong component of the identifiable signals associated with the first type of the specific particles having a relatively high abundance.

22. The method of claim 21, wherein the second measurement at the second configuration is adapted to effectively measure a relatively weak component of the identifiable signals associated with the second type of the specific particles having a relatively low abundance.

23. The method of claim 22, wherein combining the first and second output signals comprises scaling the first output signal to a scale associated with the second configuration such that the based on the second configuration, the weak component is effectively measured and the strong component is effectively represented based on the scaling of the effectively measured value from the first configuration.

24. The method of claim 23, wherein the scaling of the strong component allows effective representation of both weak and strong components when a dynamic range associated with the detector is limited and would not be able to measure the strong component at the second configuration.

25. The method of claim 24, wherein the detector is a charge-coupled device and the first configuration comprises a short exposure duration T1 selected to effectively measure the strong component of the identifiable signals.

26. The method of claim 25, wherein the second configuration comprises a long exposure duration T2 selected to effectively measure a weak component of the identifiable signals.

27. The method of claim 26, wherein the scaling of the first output signal comprises multiplying the first output signal value by a ratio T2/T1.

28. The method of claim 24, wherein the detector is a charge multiplier and the first configuration comprises a low operating voltage V1 selected to effectively measure the strong component of the identifiable signals.

29. The method of claim 28, wherein the second configuration comprises a high operating voltage V2 selected to effectively measure a weak component of the identifiable signals.

30. The method of claim 29, wherein the scaling of the first output signal comprises determining the scaled value N1′ of the first output signal based on a relationship log(N1′)=m log(V2/V1) where m represents a slope of a curve obtained by plotting the multiplier's gain versus the voltage in a log-log manner.

31. The method of claim 30, wherein the charge multiplier comprises a photomultiplier tube.

32. The method of claim 30, wherein the charge multiplier comprises a charge intensifier.

33. A method extending the effective dynamic range of a detector that measures detectable signals from a sample undergoing a biological analysis wherein the detectable signals comprise two or more components representative of two or more components of the sample, the method comprising:
obtaining a first output signal from the detector operated at a first configuration that allows effective measurement of a first component of the detectable signals;
obtaining a second output signal from the detector operated at a second configuration that allows effective measurement of a second component of the detectable signals wherein the second configuration is such that the first component of the detectable signals would fall outside the detector's dynamic range at the second configuration; and
scaling the first output signal to a scale associated with the second configuration wherein the amount of scaling depends on the first and second configurations and wherein the scaled first output signal allows representation of the first output signal at the second configuration thereby extending the effective dynamic range of the detector and wherein such extension of the effective dynamic range allows improved characterization of the sample having a relatively large range of relative abundances of the two or more components.

34. The method of claim 33, wherein the first configuration is adapted to effectively measure a strong component of the detectable signals.

35. The method of claim 34, wherein the second configuration is adapted to effectively measure a weak component of the detectable signals.

36. The method of claim 35, wherein scaling the first output signal allows representation of both weak and strong components when the dynamic range associated with the detector is limited and would not be able to measure the strong component at the second configuration.

37. The method of claim 36, wherein the detector is a charge-coupled device and the first configuration comprises a short exposure duration T1 selected to effectively measure the strong component of the detectable signals.

38. The method of claim 37, wherein the second configuration comprises a long exposure duration T2 selected to effectively measure a weak component of the detectable signals.

39. The method of claim 38, wherein the scaling of the first output signal comprises multiplying the first output signal value by a ratio T2/T1.

40. The method of claim 36, wherein the detector is a charge multiplier and the first configuration comprises a low operating voltage V1 selected to effectively measure the strong component of the detectable signals.

41. The method of claim 40, wherein the second configuration comprises a high operating voltage V2 selected to effectively measure a weak component of the detectable signals.

42. The method of claim 41, wherein the scaling of the first output signal comprises determining the scaled value N1′ of the first output signal based on a relationship log(N1′)=m log(V2/V1) where m represents a slope of a curve obtained by plotting the multiplier's gain versus the voltage in a log-log manner.

43. The method of claim 42, wherein the charge multiplier comprises a photomultiplier tube.

44. The method of claim 42, wherein the charge multiplier comprises a charge intensifier.
